(19) United States
(12) Patent Application Publication
(10) Pub. No.: US 2014/0199275 A1
(43) Pub. Date: Jul. 17, 2014

(54) TREATING TEAR FILM DISORDERS WITH MESENCHYMAL STEM CELLS

(71) Applicant: The Regents of the University of California, Oakland, CA (US)

(72) Inventors: Christopher J. Murphy, Davis, CA (US); Paul Russell, Davis, CA (US); Dori L. Borjesson, Davis, CA (US); Joshua Wood, Vacaville, CA (US)

(73) Assignee: THE REGENTS OF THE UNIVERSITY OF CALIFORNIA, Oakland, CA (US)

(21) Appl. No.: 14/119,875

(22) PCT Filed: May 31, 2012
(86) PCT No.: PCT/US2012/040228

§ 371 Date: Mar. 27, 2014

Related U.S. Application Data
(60) Provisional application No. 61/492,222, filed on Jun. 1, 2011.

(51) Int. Cl. A61K 35/28; A61M 5/00

(52) U.S. Cl. CPC A61K 35/28; A61M 5/00
USPC 424/93.7; 604/272

(57) ABSTRACT

This invention provides methods of preventing, reducing or inhibiting one or more symptoms of a tear film disorder by administration to a subject mesenchymal stem cells.

### CROSS-REFERENCE TO RELATED APPLICATIONS

This application is a U.S. national phase under 35 U.S.C. §371 of International Application No. PCT/US2012/040228, filed on May 31, 2012, which claims the benefit under 35 U.S.C. §119(e) of U.S. Provisional Application No. 61/492,222, filed on Jun. 1, 2011, all of which are hereby incorporated herein by reference in their entirety for all purposes.

### FIELD OF THE INVENTION

The present invention relates to preventing, delaying, reducing or inhibiting tear film disorders (e.g., tear evaporation and/or tear production insufficiency) leading to dry eye diseases (e.g., keratoconjunctivitis sicca-KCS and/or meibomian gland dysfunction (MGD)) in a subject by the engraftment of mesenchymal stem cells (MSC).

### BACKGROUND OF THE INVENTION

Dry eye diseases (DED) impact 3.5-33.7% of people globally and are a primary reason patients seek eye care [Abderrahim, et al., Mamm. Genome, (1998) 9(8):673-675; Reddy, et al., Cornea (2004) 23(8):751-761; Lin, et al., Arch Ophthalmol (2010) 128(5):619-623; and Yu, et al., Cornea (2011) 30(4):379-87]. Significant morbidity in the form of superficial corneal erosions and corneal ulcers dramatically reduces the quality of life among patients and can result in decreased visual acuity [Abderrahim, et al., supra; Reddy, et al., supra; and Lin, et al., supra]. The costs of DED are not only humanitarian; the overall financial burden of DED is approximately $3.84 billion annually in the United States alone [Yu, et al., supra]. The underlying causes of DED are frequently due to an autoimmune or inflammatory disorder and treatment often includes the use of topical cyclosporine (an immunosuppressant) and corticosteroids (an anti-inflammatory agent) [Reddy, et al., supra].

The co-incidence of systemic autoimmune or inflammatory diseases in patients with DED is as high as 71.8% [Lin, et al., supra; Moss, et al., Arch Ophthalmol (2004) 122(3):369-373]. Keratoconjunctivitis sicca (KCS) is one form of DED that reduces or eliminates tear production by the lacrimal gland [Nguyen, et al., Experimental Eye Research (2009) 88(3):398-409]. The underlying cause of KCS is frequently Sjögren's syndrome, an autoimmune disease that results in destruction of the lacrimal gland [Nguyen, et al., supra]. Rheumatoid arthritis (RA) is another automimmune disease that is frequently associated with DED. In RA related DED cases, treatment using systemic and topical immunosuppressants is recommended by the American Academy of Ophthalmology [Reddy, et al., supra; Lin, et al., supra].

Systemic autoimmune diseases including RA affect the joints and synovium of patients in addition to the lacrimal glands. Approximately 1.3 million adults in the U.S. have been diagnosed with RA [Helmick, et al., Arthritis & Rheumatism (2008) 58(1):15-25]. Excluding the costs of co-morbid factors, the annual U.S. expenditure in terms of health care costs and lost productivity is approximately $14.8 billion [Helmick, et al., supra; Boonen, et al., Clin Exp Rheumatol (2009) 27(S55): S112-117]. The significant morbidities and mortality associated with joint autoimmune disorders dramatically reduce quality of life and can result in premature death [Boonen, et al., supra; Gabriel, et al., Arthritis Research & Therapy (2009) 11(3):229]. The current treatment regiments for RA addresses initial symptoms with non-steroidal anti-inflammatory drugs (NSAIDs). As disease progression occurs, so does the nature of the treatment with the use of chemotherapeutic agents such as cyclophosphamide [Isaacs, Nat Rev Immunol, (2010) 10(8):605-611]. Despite these advances, significant therapeutic gaps remain in the ability to treat and prevent localized and systemic autoimmune diseases.

One therapeutic approach for autoimmune diseases and disorders is treatment using stem cell therapy. Mesenchymal stem cells (MSCs) are an adult stem cell source that can be derived from a host of anatomic origins including bone marrow [Zucconi, et al., Stem Cells and Development (2010) 19(3):395-402; Colletti, et al., Stem Cell Research (2009) 2(2):125-138] and, with less impact on the donor, adipose tissue [Neupane, et al., Tissue Engineering Part A, (2008) 14(6):1007-1015; Vieira, et al., Cell Transplantation (2010) 19:279-289; and Kern, et al., Stem Cells (2006) 24(5):1294-1301]. Adipose derived (Ad) MSCs have been previously isolated and characterized from several animal models including canines [Vieira, et al., supra, Chung, et al., Res Vet Sci. (2010) November 12, PMID:21075407; Martinello, et al., Res Vet Sci. (2010) August 21, PMID:20732703]. MSCs are characterized by their ability to differentiate into osteocytes, chondrocytes, and adipocytes and the cluster of differentiation (CD) markers they express. While debate remains over which CD markers classify MSCs, a consensus is that MSC should at a minimum express CD44 and CD90, and should not express CD34, CD45, CD80, CD86 or MHC-II [Neupane, M., et al., supra; Vieira, et al., supra; Djouad, et al., Nat Rev Rheumatol (2009) 5(7):392-399; and Chamberlain, et al., Stem Cells (2007) 25(11):2739-2749].

Ad-MSCs are being investigated for the treatment of numerous disorders and diseases including skin regeneration, chemotherapy, spinal cord injury, diabetes, and immune disorders in preclinical applications [Djouad, et al., supra; Zuk, et al., Mol. Biol. Cell (2010) 21(11):1783-1787; Ben-Ami, et al., Autoimmun Rev. (2011) January 20, PMID:21256250]. In addition to preclinical investigations, adipose derived stem cells and MSCs from other sources are currently being used in clinical aspects ranging from bone grafting to treatment of immune disorders including Crohn's and graft versus host disease [Zuk, et al, supra; Ben-Ami, et al., supra; Singer, et al., Annual Review of Pathology: Mechanisms of Disease, (2011) 6(1):457-478; Lee, et al., Clin Pharmacol Ther (2007) 83(5):723-730]. The immunomodulatory ability of MSCs has been shown to impact both innate and adaptive immunity [Ben-Ami, et al., supra]. MSCs have been shown to inhibit T-cell proliferation, and with some controversy, B-cell proliferation [Ben-Ami, et al., supra]. MSCs have also been shown to down regulate MHC class II and inhibit the maturation of dendritic cells as well as the differentiation of hematopoietic stem cells (HSCs) into dendritic cells [Ben-Ami, et al., supra].

### SUMMARY OF THE INVENTION

The present invention provides methods of preventing, delaying, reducing or inhibiting a tear film disorder (e.g., resulting from excessive tear evaporation and/or insufficient tear production) in a subject in need thereof comprising transplanting or engrafting mesenchymal stem cells (MSC). In some embodiments, the transplanted or engrafted MSCs exert an immunosuppressive effect on immune mediators of target tissue destruction to prevent, delay, reduce or inhibit the destruction or further destruction of the target tissue (e.g., the lacrimal gland and/or meibomian glands and/or goblet cells of the conjunctiva), thereby alleviating the symptoms of the tear film disorder or dry eye disease in the subject. Engrafting or transplanting MSCs into a subject to counteract the symptoms of tear film disorder or dry eye disease is a prophylactic and therapeutic strategy to counteract the symptoms of a tear film disorder or a dry eye disease that can be used instead of or in addition to the administration of pharmacological agents.

With respect to the embodiments of the methods, in some embodiments, the MSCs are adipose-derived mesenchymal stem cells (Ad-MSC). In various embodiments, the Ad-MSCs can be characterized by the surface expression of CD44, CD5, and CD90 (Thy-1); and by the non-expression of CD34, CD45, MHC class II, CD3, CD80, CD86, CD 18 and CD49d.

In some embodiments, the MSCs are derived from a non-adipose tissue. In some embodiments, the non-adipose tissue is selected from the group consisting of bone marrow, liver, lacrimal gland, and peri-ocular tissues. In some embodiments, the non-adipose tissue is selected from the group consisting of liver and peri-ocular tissues. In some embodiments, the MSCs are derived from liver. In some embodiments, the MSCs are not derived from and/or do not comprise lacrimal gland tissue. In some embodiments, the MSCs are non-haematopoietic stem cells derived from bone marrow (i.e., do not express CD34 or CD45). In some embodiments, the MSCs express CD44 and CD90 and do not express CD34, CD45, CD80, CD86 or MHC-II.

In some embodiments, the MSCs are autologous to the subject (i.e., from the same subject). In some embodiments, the MSCs are syngeneic to the subject (i.e., from a subject having an identical or closely similar genetic makeup). In some embodiments, the MSCs are allogeneic to the subject (i.e., from a subject of the same species). In some embodiments, the MSCs are xenogeneic to the subject (i.e., from a subject of a different species). In one embodiment, adipose derived MSCs that are autologous to the subject are administered. In one embodiment, adipose derived MSCs that are allogeneic to the subject are administered.

In various embodiments, the tear film disorder or dry eye disease is an aqueous deficient disorder or dry eye disease (i.e., the tear film disorder is characterized by a deficiency in the aqueous component of the tear film). In some embodiments, the dry eye disease is keratoconjunctivitis sicca (KCS).

In some embodiments, the tear film disorder or dry eye disease is an evaporative disorder or dry eye disease (i.e., due to accelerated or excessive tear evaporation). In some embodiments, the dry eye disease is a Meibomian Gland Dysfunction (MGD). In some embodiments, the MGD is selected from the group consisting of posterior blepharitis, meibomian gland disease, meibomitis, meibomianitis, and meibomian keratoconjunctivitis.

In some embodiments, the tear film disorder or dry eye disease results from damage or destruction to the goblet cells of the conjunctiva. In such cases, the tear film disorder may be characterized by a deficiency in the mucous (glycosaminoglycan) component of the pre-corneal tear film.

Some subjects may suffer concurrently from an aqueous deficient dry eye disease and an evaporative eye disease. Some subjects may suffer from a tear film disorder that is characterized by a combination deficiency of one or more of the 3 components of the pre-corneal tear film (aqueous and/or Meibomian gland (i.e., meibum) secretions and/or mucous/glycosaminoglycan secretions).

Engraftment or transplantation of MSCs alleviates one or more symptoms of dry eye disease in the subject. In some embodiments, the alleviated symptom is selected from the group consisting of loss of the lacrimal gland tissue, loss of meibomian gland tissue, loss of goblet cells, and damage to the cornea (thickening of the corneal surface, corneal erosion, corneal ulceration, corneal neovascularization, corneal scarring, corneal thinning, and corneal perforation). In some embodiments, the alleviated symptom is insufficient, unproductive or non-lubricating tear production. Engraftment or transplantation of MSCs can stably increase tear production in the treated eye, e.g., in comparison to tear production in the same eye before engraftment or transplantation of MSCs. In some embodiments, the alleviated symptom is excessive evaporation of the tear film. In some embodiments, the alleviated symptom is eye irritation (e.g., dryness, burning, sandy-gritty sensations, itching, stinging, fatigue, pain, redness, pulling sensations).

In some embodiments, destruction or further destruction to the lacrimal gland and/or the meibomian glands and/or goblet cells of the conjunctiva are prevented, reduced or inhibited. In some embodiments, the cell number, mass and/or functionality of the lacrimal gland and/or meibomian glands and/or goblet cells of the conjunctiva are increased. In some embodiments, tear production is increased or elevated (e.g., measurably e.g., by Schirmer tear test, 10%, 25%, 50%, 1-fold, 2-fold, 5-fold, 10-fold, or more) after engrafting or transplanting in the subject the mesenchymal stem cells (MSC), e.g., in comparison to tear production in the same eye before engraftment or transplantation of MSCs.

In various embodiments, the MSCs are administered systemically, and can exert a systemic immunosuppress effect. In some embodiments, the MSCs are engrafted or transplanted sufficiently close to the target tissue, e.g., the lacrimal gland and/or the meibomian glands, to allow for the immunosuppressive factors secreted by the MSCs to prevent, reduce, or inhibit immune-mediated destruction of the target tissue, e.g., the lacrimal gland and/or the meibomian glands, respectively. In some embodiments, the MSCs are engrafted or transplanted peri-ocularly, subconjunctivally, in and/or around the conjunctiva, or into the stroma of the eyelid. In subjects suffering dry eye disease due to insufficient function, damage or destruction to the lacrimal gland, the MSCs may be administered, engrafted or transplanted peri-ocularly or peri-lacrimally. In subjects suffering dry eye disease due to insufficient function, damage or destruction to the meibomian glands, the MSCs may be administered, engrafted or transplanted into the stroma of the eyelid, e.g., at or near the lid margin.

In some embodiments, at least 0.25×10⁶ MSCs are administered to the subject. For example, in various embodiments, at least about 0.25×10⁶ MSCs, 0.5×10⁶ MSCs, 1×10⁶ MSCs, 5×10⁶ MSCs, 1×10⁷ MSCs, 5×10⁷ MSCs, 1×10⁸ MSCs or 5×10⁸ MSCs are administered to the subject.

As needed or appropriate, the MSCs can be administered once or multiple times. In some embodiments, the MSCs are administered to the subject multiple times. For example, in various embodiments, the MSCs are administered to the subject, e.g., daily, weekly, monthly, every other month, every third or fourth month, twice annually, annually, e.g., until a population of MSCs is engrafted or transplanted into the subject sufficient to prevent, delay, reduce or inhibit dry eye disease.

In some embodiments, the subject is a mammal. In some embodiments, the subject is a human. In some embodiments, the subject is a canine.

In some embodiments, the dry eye disease is caused by or is secondary to or is a symptom of an autoimmune disease. In some embodiments, the subject has an autoimmune disease or has a predisposition to developing an autoimmune disease. For example, the subject may have, be diagnosed with, or have a predisposition to developing rheumatoid arthritis, Wegener's granulomatosis, systemic lupus erythematosus (SLE), Sjögren's syndrome, scleroderma, primary biliary cirrhosis, diabetes or Vogt-Koyanagi-Harada Syndrome (VKH Syndrome). In some embodiments, the subject has an autoimmune disease or has a predisposition to developing Stevens-Johnson syndrome.

In a related but distinct aspect, the invention provides a cell delivery device, comprising a needle and a stop positioned along the length of the needle. In some embodiments, the external surface of the needle is demarcated by cross-wise scores. In some embodiments, the position of the stop is adjustable along the length of the needle. The needle has an inner lumen of sufficient diameter for the delivery of MSCs to a desired target tissue, with minimal or reduced shearing or damage to the MSCs.

In another aspect, the invention provides a kit comprising mesenchymal stem cells (MSCs) and the cell delivery device described herein. In some embodiments, the MSCs are adipose-derived mesenchymal stem cells (Ad-MSC).

Further embodiments are as described herein.

### DEFINITIONS

The terms “individual,” “patient,”, “subject” interchangeably refer to a mammal, for example, a human, a non-human primate, a domesticated mammal (e.g., a canine or a feline), an agricultural mammal (e.g., equine, bovine, ovine, porcine), or a laboratory mammal (e.g., rattus, murine, lagomorpha, hamster).

As used herein, the terms “treating” and “treatment” refer to delaying the onset of, retarding or reversing the progress of, reducing the severity of, or alleviating or preventing either the disease or condition to which the term applies (i.e., dry eye syndromes or dry eye diseases), or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

The term “effective amount” or “therapeutically effective amount” refers to the amount of an active agent sufficient to induce a desired biological result (e.g., prevention, delay, reduction or inhibition of one or more symptoms of a dry eye disease or a dry eye syndrome). That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “dry eye diseases” refers to a group of conditions each characterized by decreased tear production or increased tear film evaporation. See, e.g., Ocul Surf. (2007) 5(2):75-92; Latkany, Curr Opin Ophthalmol. (2008) 19(4):287-91.

“Keratoconjunctivitis sicca (KCS),” “keratitis sicca,” “xerophthalmia,” or “dry eye syndrome (DES)” is a dry eye disease caused by either decreased tear production or increased tear film evaporation. Keratoconjunctivitis sicca is usually due to inadequate tear production. The aqueous tear layer is affected, resulting in aqueous tear deficiency (ATD) or lacrimal hyposecretion. In subjects with KCS, the lacrimal gland does not produce sufficient tears to keep the entire conjunctiva and cornea covered by a complete layer. Symptoms include eye irritation (e.g., dryness, burning, sandy-gritty sensations, itching, stinging, fatigue, pain, redness, pulling sensations), stingy discharge from the eyes, and the production of non-lubricating tears. In advanced cases, the epithelium, e.g., of the upper eyelid may undergo squamous metaplasia and loss of goblet cells. Some severe cases may result in thickening of the corneal surface, corneal erosion, punctate keratopathy, epithelial defects, corneal ulceration (sterile and infected), corneal neovascularization, corneal scarring, corneal thinning, and/or even corneal perforation.

Meibomian Gland Dysfunction (MGD) refers to a chronic, diffuse abnormality of the meibomian glands, commonly characterized by terminal duct obstruction and/or qualitative/quantitative changes in the glandular secretion. MGD may result in alteration of the tear film, symptoms of eye irritation, clinically apparent inflammation, and ocular surface disease. Nichols, et al., Investigative Ophthalmology & Visual Science (2011) 52(4):1922-1929. MGD is considered an “evaporative” dry eye condition, oftentimes due to a loss of amount and/or integrity of the lipid component of the precorneal tear film. MGD conditions include without limitation posterior blepharitis, meibomian gland disease, meibomitis, meibomianitis, and meibomian keratoconjunctivitis. The pathophysiological mechanisms can be broadly categorized into 1. low delivery of meibum (due to obstruction or hyposecretion—either primary of secondary in nature) and 2. high delivery of meibum (either primary or secondary hypersecretion). See, e.g., Nichols, et al., supra; Geerling, et al., Investigative Ophthalmology & Visual Science (2011) 52(4):2050-2064; and Nichols, Investigative Ophthalmology & Visual Science (2011) 52(4):1917-1921.

The terms “increasing,” “promoting,” “enhancing” refers to increasing the mass, bulk, cell number and/or functionality of the lacrimal gland in a subject by a measurable amount using any method known in the art. The mass, bulk, cell number and/or functionality of the lacrimal gland is increased, promoted or enhanced if the mass, bulk, cell number and/or functionality of the lacrimal gland is at least about 10%, 20%, 30%, 50%, 80%, or 100% increased in comparison to the mass, bulk, cell number and/or functionality of the lacrimal gland prior to administration of mesenchymal stem cells (MSCs). In some embodiments, the mass, bulk, cell number and/or functionality of the lacrimal gland is increased, promoted or enhanced by at least about 1-fold, 2-fold, 3-fold, 4-fold, or more in comparison to the mass, bulk, cell number and/or functionality of the lacrimal gland prior to administration of the MSCs. In various embodiments, “increasing,” “promoting,” “enhancing” refers to increasing or elevating tear production. In various embodiments, tear production is increased, promoted or enhanced if the measured tear production, e.g., by Schirmer tear test, is at least about 10%, 20%, 30%, 50%, 80%, 100%, 1-fold, 2-fold, 3-fold, 4-fold, or more, increased in comparison to the measured tear production prior to administration of mesenchymal stem cells (MSCs).

The terms “inhibiting,” “reducing,” “decreasing” with respect to the dry eye disease being treated (e.g., KCS) refers to inhibiting one or more symptoms of the dry eye disease in a subject by a measurable amount using any method known in the art (e.g., alleviated or mitigated symptoms can include one or more of loss of the lacrimal gland tissue, loss of goblet cells, damage to the cornea (e.g., thickening of the corneal surface, corneal erosion, corneal ulceration, corneal neovascularization, corneal scarring, corneal thinning, and corneal perforation); insufficient, unproductive or non-lubricating tear production; and/or eye irritation (e.g., dryness, burning, sandy-gritty sensations, itching, stinging, fatigue, pain, redness, pulling sensations)). The one or more symptoms of dry eye disease are inhibited, reduced or decreased if the measurable parameter of the one or more symptoms is at least about 10%, 20%, 30%, 50%, 80%, or 100% reduced in comparison to the measurable parameter of the one or more symptoms prior to administration of the MSCs. In some embodiments, the measurable parameter of the one or more symptoms is inhibited, reduced or decreased by at least about 1-fold, 2-fold, 3-fold, 4-fold, or more in comparison to the measurable parameter of the one or more symptoms prior to administration of the MSCs.

The term “mesenchymal stem cells” refers to stem cells defined by their capacity to differentiate into bone, cartilage, and adipose tissue. With respect to cell surface markers, MSCs generally express CD44 and CD90, and should not express CD34, CD45, CD80, CD86 or MHC-II.

As used herein, “administering” refers to local and systemic administration, e.g., including enteral and parenteral administration. Routes of administration for the MSCs that find use in the present invention include, e.g., administration as a suppository, intravenous (“iv”), intraperitoneal (“ip”), intramuscular (“im”), intralesional, intranasal, or subcutaneous (“sc”) administration. Administration may also be local to the target or damaged tissue, e.g., local to the lacrimal gland or the meibomian glands. For example, the MSCs may be administered, engrafted or transplanted peri-ocularly, intra-lacrimally, peri-lacrimally, subconjunctivally, into the eyelid stroma, e.g., at the lid margin, into or around the meibomian glands. Administration can be by any appropriate route such that the immunosuppressive agents secreted by the MSCs prevent, reduce or inhibit destruction or damage to the target tissue, e.g., the lacrimal gland and/or the meibomian glands and/or goblet cells of the conjunctiva. Parenteral administration includes, e.g., intravenous, intramuscular, intra-arteriole, intraventricular, intradermal, subcutaneous, intraperitoneal, and intrarectal.

The terms “systemic administration” and “systemically administered” refer to a method of administering a compound or composition to a mammal so that the MSCs delivered to sites in the body, including the targeted site of pharmacological action, via the circulatory system. Systemic administration includes, e.g., intravenous, intra-arteriole, intraventricular intradermal, subcutaneous, intraperitoneal, and rectal administration.

The term “co-administer” and “co-administering” and variants thereof refer to the simultaneous presence of two or more active agents in the blood of an individual. The active agents that are co-administered can be concurrently or sequentially delivered.

The phrase “cause to be administered” refers to the actions taken by a medical professional (e.g., a physician), or a person controlling medical care of a subject, that control and/or permit the administration of the agent(s)/compound(s)/cell(s) at issue to the subject. Causing to be administered can involve diagnosis and/or determination of an appropriate therapeutic or prophylactic regimen, and/or prescribing particular agent(s)/compounds/cell(s) for a subject. Such prescribing can include, for example, drafting a prescription form, annotating a medical record, and the like.

### BRIEF DESCRIPTION OF THE DRAWINGS

FIGS. 1A and B illustrate in vivo fluorescence of Ad-MSC persists up to 3 weeks following engraftment or transplantation into the lacrimal region. Representative images demonstrate the persistence of the fluorescent signal in the right peri-lacrimal region of injected animals compared to vehicle injected, left peri-lacrimal regions (A) for up to 3 weeks after injection. The average signal intensity (n=3) was significantly brighter than left peri-lacrimal controls immediately following injection (p<0.01) and for the two subsequent weeks (p<0.05) following injection. Average signal intensity was also significantly brighter (p<0.05) than left peri-lacrimal controls immediately following the 2nd injection of DiD labeled Ad-MSC (B). All data are presented as mean±SEM.

FIGS. 2A and B illustrate in vivo fluorescence of Ad-MSC persists up to 2 weeks following engraftment or transplantation into the stifle region. Representative images demonstrate the persistence of the fluorescent signal in the right medial stifle region of injected animals compared to vehicle injected, left medial stifle regions (A) for up to 2 weeks after injection. The combined average signal intensity (n=3) from both the lateral and medial regions of the right stifle was significantly brighter than combined left stifle controls one week after injection (p<0.001) (B). All data are presented as mean±SEM.

FIGS. 3A-D illustrate Ad-MSC engrafted in the peri-lacrimal and 3rd eye lid (EL) gland regions. Ex vivo imaging over 4 weeks following the engraftment or transplantation of labeled MSC demonstrates persistence of the cells in representative images of the lacrimal region and region of the 3rd EL gland (A). Heat maps demonstrate localization of the cells around the lacrimal and 3rd EL glands compared to vehicle injected, left side controls (B). ANOVA results revealed statistically significant differences (p<0.05) between the treatment groups for both lacrimal and 3rd EL samples but there was insufficient statistical power to elucidate the level significance between individual groups using paired sample analysis (C,D). Heat map signal intensity is presented as 10⁶ phot/cm²/s and all data are presented as mean±SEM.

FIGS. 4A-D illustrate Ad-MSC engrafted in the cartilage and joint capsule following engraftment or transplantation. Ex vivo imaging over 4 weeks following the engraftment or transplantation of labeled MSC demonstrates persistence of the cells in representative images of the cartilage region and joint capsule (A). Heat maps demonstrate localization of the signal to the compared to vehicle injected, left side controls (B). Engraftment of labeled MSC was found in the right cartilage and joint capsule regions of directly injected animals compared to the respective regions of the left legs and both right and left respective regions of animals receiving lacrimal injections. Average signal intensity in the cartilage surrounding the stifle joint is more than 7 times greater than the average signal intensity from the left stifle joint cartilage and nearly 70 times greater than the average signal intensities from cartilage from the left and right stifle joints of peri-ocular injected animals (C). Average signal intensity in the joint capsule of the right stifle joint of intra-articular injected animals is more than 150 times greater than the joint capsule of either the left stifle joint of intra-articular injected or either stifle joint of peri-ocular injected animals (D). Heat map signal intensity is presented as 10⁶ phot/cm²/s and all data are presented as mean±SEM.

FIGS. 5A-B illustrate Ad-MSC migration and engraftment to the thymus following injection. Ex vivo imaging over 4 weeks following the engraftment or transplantation of labeled MSC demonstrates migration of the cells in heat map images of the thymus (A). Significant engraftment (p<0.05) was found in the stifle injected animals compared to peri-ocular injected animals (B). Heat map signal intensity is presented as 10⁶ phot/cm²/s and all data are presented as mean±SEM.

FIGS. 6A-B illustrate Ad-MSC migration and engraftment to the GI tract following injection. Ex vivo imaging over 4 weeks following the engraftment or transplantation of labeled Ad-MSC demonstrates migration of the cells in heat map images of the tongue, stomach, duodenum, jejunum, ileum, and colon (A). Significant engraftment (*, # p<0.05; **, ## p<0.01) was found in the stomach, duodenum, jejunum, and colon of stifle injected animals compared to either peri-ocular injected (*) or control (#) animals (B). Heat map signal intensity is presented as 10⁶ phot/cm²/s and all data are presented as mean±SEM.

FIG. 7 illustrates the results of mixed leukocyte reactions (MLRs). The horizontal black line represents the regular proliferation rate of peripheral blood mononuclear cells (PBMCs) isolated from whole blood of canine subjects. The proliferation rate of PBMCs was measured as a demonstration of an immune response to a presented antigen (e.g., mesenchymal stem cells) prior to and after injection of mesenchymal stem cells (MSCs). The positive control was PBMCs mixed with irradiated allogeneic PBMCs, wherein the irradiated allogeneic PBMCs are incapable of cell division. The negative control was PBMCs mixed with irradiated autologous PBMCs. In one test sample, the proliferation of PBMCs mixed with irradiated allogeneic MSCs was measured. In another test sample, the proliferation of PBMCs, mixed with irradiated allogeneic PBMCs and irradiated allogeneic MSCs, was measured. With respect to the positive control, proliferation of PBMCs was observed both pre and post injection, but was subdued after injection of MSCs. With respect to the negative control, the canine subjects did not respond to their own PBMCs and thus the levels are similar to the normal PBMC proliferation rate. When mixed with PBMCs, irradiated allogeneic MSCs did not induce proliferation. PBMCs mixed with irradiated allogeneic MSCs and an inducer of proliferation (i.e., irradiated allogeneic PBMCs) resulted in increased PBMC proliferation prior to the MSC injection course. Interestingly, after the injection course, there is a significantly (p<0.05) lower PBMC proliferation rate even with stimulation. The results demonstrate that the MSCs had a systemic immunomodulatory effect on the dogs.

FIGS. 8A-B illustrate a needle for use in administering stem cells. As depicted, the needle comprises scores demarcating distance and an adjustable stop to facilitate delivery of stem cells to a desired location. (A) illustrates a stop with a blunt end surface; (B) illustrates a stop with a rounded end surface.

FIGS. 9A-B illustrate treated eye in canine subject A. at day −49 (first visit of subject to UC Davis Veterinary Medical Teaching Hospital (VMTH); and B. at day 42 (Week 6 post first injection of MSCs). The eye shows that the canine subject suffered no adverse events. Moreover, tear production levels, as measured by Schirmer tear tests, remained elevated.

### DETAILED DESCRIPTION

### 1. Introduction

The present invention is based, in part, on the discovery that mesenchymal stem cells (MSCs) find use in the treatment and prevention of tear film disorders (e.g., tear evaporation and/or tear production insufficiency) leading to dry eye diseases, including keratoconjunctivitis sicca (KCS) and Meibomian Gland Dysfunction (MGD). MSCs exert a profound inhibitory effect on proliferation of T cells, B cells, dendritic cells, natural killer (NK) cells and neutrophils, in vitro and in vivo. See, e.g., Ben-Ami, et al., Autoimmun Rev. (2011) January 20, PMID:21256250]; and Singer, et al., Annual Review of Pathology: Mechanisms of Disease, (2011) 6(1):457-478. Therefore, MSCs find use to dampen immune-mediated diseases. The present invention utilizes MSCs to prevent, delay, reduce or inhibit immune-mediated destruction or damage or further destruction or damage of the lacrimal gland and/or meibomian glands and/or goblet cells of the conjunctiva, preserving the ability of the remaining and intact lacrimal gland tissue and/or meibomian gland tissue and/or goblet cells of the conjunctiva for lubricating tear production, thereby alleviating symptoms of dry eye syndrome. Transplantation or engraftment of MSCs further can stably increase or elevate tear production in the treated eye.

Autoimmune diseases including Keratoconjunctivitis sicca (KCS) and Rhuematoid arthritis (RA) affect millions of people worldwide with an economic impact measured in the billions of dollars. The unique immunomodulatory properties of MSCs provide a promising therapeutic for the application of stem cell therapy to treat autoimmune disorders, including KCS. MSCs are being investigated in the treatment of RA but the application of MSCs in the treatment of KCS prior to the present application had yet to be investigated. Furthermore, the persistence and migration potential of subcutaneous MSC engraftment or transplantation had yet to be investigated with in vivo imaging techniques, e.g., in the canine model. Data provided herein provide the in vivo time course of persistence and migration of MSCs following engraftment or transplantation into the peri-lacrimal region. Also presented are findings on the preferential engraftment of MSCs throughout the gastrointestinal tract as well as the thymus compared to over 65 tissues following intra-articular engraftment or transplantation. These results demonstrate that MSC therapy for dry eye diseases, including KCS and MGD, can be translated to animal and human clinical trials and accelerate the application of MSC based therapies in clinical dry eye disease (DED) applications. These data also provide evidence for the treatment of autoimmune and inflammatory disorders using MSCs.

### 2. Subjects Amenable to Treatment

Patients amenable to treatment include individuals at risk of disease but not showing symptoms, as well as patients presently showing symptoms of a dry eye disease. The dry eye disease may be an aqueous-deficient dry eye disease (due to insufficient tear production) or an evaporative dry eye disease (e.g., excessive tear film evaporation, e.g., due to a loss of amount and/or integrity of the lipid component of the precorneal tear film), or have components of both an aqueous-deficient dry eye disease and an evaporative dry eye disease.

In various methods of treatment, the subject may already exhibit symptoms of disease or be diagnosed as having a dry eye disease. For example, the subject may be exhibiting one or more symptoms, including eye irritation (e.g., dryness, burning, sandy-gritty sensations, itching, stinging, fatigue, pain, redness, pulling sensations), stingy discharge from the eyes, and the production of non-lubricating tears; squamous metaplasia and loss of goblet cells in the epithelium; and/or damage to the cornea (e.g., thickening of the corneal surface, corneal erosion, punctate keratopathy, epithelial defects, corneal ulceration (sterile and infected), corneal neovascularization, corneal scarring, corneal thinning, and/or corneal perforation). In such cases, administration of MSCs can reverse or delay progression of and or reduce the severity of disease symptoms.

The effectiveness of treatment can be determined by comparing a baseline measure of a parameter of disease before administration of the MSCs is commenced to the same parameter one or more timepoints after MSCs have been administered. Illustrative parameters that can be measured include without limitation stabilization and/or increase in the bulk, mass, cell number and/or functionality of the lacrimal gland and/or the meibomian glands; tear production and/or tear composition; examination of the corneal and/or conjunctival tissues; and/or reporting by the patient. Increased bulk, mass, cell number and/or functionality of the lacrimal gland and/or the meibomian glands, increased tear production and tear composition having normal salt and/or lipid concentrations/compositions, and/or stabilized and/or improved corneal and/or conjunctival tissues is an indicator that the treatment is effective.

For the purposes of prophylaxis, the subject may be asymptomatic but have a risk or predisposition to developing a dry eye disease. For example, the subject may have an autoimmune disease that causes or is associated with the development of a dry eye disease. In such cases, administration of MSCs can prevent or delay onset of disease or progression of dry eye disease into later stages of disease, and/or reduce the severity of the disease once present.

In some embodiments, the subject has an autoimmune disease. For example, the subject may have an autoimmune disease the result in immune-mediated destruction of the lacrimal gland and/or the meibomian glands and/or goblet cells of the conjunctiva. Subjects who have or are diagnosed with an autoimmune disease that causes or is associated with symptoms of dry eye disease, including without limitation, rheumatoid arthritis, Wegener's granulomatosis, systemic lupus erythematosus (SLE), Sjögren's syndrome, scleroderma, primary biliary cirrhosis, diabetes or Vogt-Koyanagi-Harada Syndrome (VKH Syndrome) are candidates for treatment or prevention of dry eye disease by administration of MSCs. In some embodiments, the subject has an autoimmune disease or has a predisposition to developing Stevens-Johnson syndrome. The subject may or may not exhibit symptoms of dry eye disease.

### 3. Administration of Mesenchymal Stem Cells (MSCs)

a. Mesenchymal Stem Cells

The bone marrow of an adult mammal is a repository of mesenchymal stem cells (MSCs). These cells are self-renewing, clonal precursors of non-hematopoietic tissues. MSCs for use in the present methods can be isolated from a variety of tissues, including bone marrow, muscle, fat (i.e., adipose), liver, peri-ocular tissues including lacrimal gland and meibomian glands, and dermis, using techniques known in the art. Illustrative techniques are described herein and reported in, e.g., Chung, et al., Res Vet Sci. (2010) November 12, PMID:21075407; Toupadakis, et al., American Journal of Veterinary Research (2010) 71(10):1237-1245. Depending on the stimulus and the culture conditions employed, these cells can form bone, cartilage, tendon/ligament, muscle, marrow, adipose, and other connective tissues.

In various embodiments, MSCs selected for use in preventing, reducing, reversing or inhibiting the immune-mediated destruction of lacrimal gland tissue and/or meibomian gland tissues and/or goblet cells of the conjunctiva secrete one or more immunosuppressive agents, including without limitation prostaglandin E2 (PGE2), human leukocyte antigen G5 (HLA-G5), hepatocyte growth factor (HGF), inducible nitric oxide synthase (iNOS), indoleamine-2,3-dioxygenase (IDO), transforming growth factor β (TGF-β), leukemia-inhibitory factor (LIF), and interleukin (IL)-10.

Generally, the MSCs useful for administration express on their cell surface CD44 and CD90 and do not express on their cell surface CD34, CD45, CD80, CD86 or MHC-II. In various embodiments, the MSCs are adipose-derived mesenchymal stem cells (Ad-MSC). Ad-MSCs can be characterized by the surface expression of CD44, CD5, and CD90 (Thy-1); and by the non-expression of CD34, CD45, MHC class II, CD3, CD80, CD86, CD 18 and CD49d. In other embodiments, the MSCs are derived from a non-adipose tissue, for example, bone marrow, liver, lacrimal gland, and peri-ocular tissues. In some embodiments, the MSCs are not derived from and/or do not comprise lacrimal gland tissue. In some embodiments, the MSCs are non-haematopoietic stem cells derived from bone marrow (i.e., do not express CD34 or CD45).

As appropriate, the MSCs can be autologous (i.e., from the same subject), syngeneic (i.e., from a subject having an identical or closely similar genetic makeup); allogeneic (i.e., from a subject of the same species) or xenogeneic to the subject (i.e., from a subject of a different species).

In various embodiments, the MSCs may be altered to enhance the viability of engrafted or transplanted cells. For example, the MSCs can be engineered to overexpress or to constitutively express Akt. See, e.g., U.S. Patent Publication No. 2011/0091430.

b. Administration

The MSCs can be administered by any appropriate route such that the immunosuppressive factors secreted by the cells exert an immune-inhibitory effect on the immune mediators of damage and/or destruction of the lacrimal gland and/or the meibomian gland tissues and/or the goblet cells of the conjunctiva that secrete mucins that are integral components of the pre-corneal tear film. The immune inhibitory effect can be local or systemic. In various embodiments, the MSCs are systemically administered. In some embodiments, the MSCs are administered locally, e.g., into the peri-ocular space, subconjunctivally, into the stroma of the eyelid.

As appropriate, the MSCs can be engrafted or transplanted into and/or around the lacrimal gland. When engrafted or transplanted in the vicinity of the lacrimal gland (i.e., peri-ocularly or peri-lacrimally), the MSCs are administered within sufficient proximity of the lacrimal gland to exert an immune-inhibitory effect on the immune mediators of damage and/or destruction of the lacrimal gland tissue. For example, the MSCs are engrafted or transplanted in sufficient proximity to the lacrimal gland such that any immunosuppressive factors secreted by the MSCs prevent, reduce or inhibit immune-mediated damage and/or destruction to the lacrimal gland. For example, the MSCs may be engrafted or transplanted within the peri-ocular space and within about 2.0 cm, 1.5 cm, 1.0 cm, 0.75 cm, 0.5 cm, 0.25 cm or 0.1 cm from the lacrimal gland. In some embodiments, the MSCs are engrafted or transplanted such that they are around the lacrimal gland (i.e., peri-lacrimally) and in contact with the gland. In some embodiments the MSC are delivered directly into the lacrimal tissue. Peri-lacrimal as well intralacrimal delivery of the MSCs can be guided by the cell delivery device, described herein. Injections into and/or around the lacrimal gland can be made using a simple standard needle (e.g., 27-20 gauge) or a small diameter spinal needle (e.g., 25 or 24 gauge). The procedure may be done alone for the treatment of KCS or in combination with other therapies, including MSC injection into the region of the meibomian glands.

As appropriate, the MSCs can be engrafted or transplanted into and/or around the meibomian glands. Providing an improved microenvironment for the cells of the meibomian glands can be achieved by the presence of MSCs in the region of the lid margin, e.g., in proximity to the meibomian glands. To achieve this, MSCs can be administered, engrafted or transplanted into the stroma of the eyelid, e.g., at the lid margin, in proximity to the meibomian glands. For example, in various embodiments, the injection can be made about 1-6 mm, for example, 2-4 mm, posterior to the lid margin. Delivery of the MSCs into the stroma of the eyelid, e.g., in the region of the lid margin, e.g., in and/or around the meibomian glands, can be guided by the cell delivery device, described herein. Injections to the lid margin can be made using a simple standard needle (e.g., 27-20 gauge) or a small diameter spinal needle (e.g., 25 or 24 gauge). The procedure may be done alone for the treatment of MGD or in combination with other therapies, including MSC injection into the region of the lacrimal gland.

In canine and feline subjects and other mammalian subjects having a third eyelid (EL) gland, the MSCs also can be engrafted or transplanted into and/or around the third eyelid (EL) gland.

Injections of MSCs can be done after local anesthetics (e.g., lidocaine, bupivacaine) have been administered. It is also possible to inject the MSCs in conjunction with local anesthetics added to the cell suspension. Injections can also be made with the subject under general anesthesia with or without the use of local anesthetic agents (e.g., lidocaine).

In various embodiments, engraftment or transplantation of the MSCs can be facilitated using a matrix or caged depot. For example, the MSCs can be engrafted or transplanted in a “caged cell” delivery device wherein the cells are integrated into a biocompatible and/or biologically inert matrix (e.g. a hydrogel or other polymer or any device) that restricts cell movement while allowing the cells to remain viable. Synthetic extracellular matrix and other biocompatible vehicles for delivery, retention, growth, and differentiation of stem cells are known in the art and find use in the present methods. See, e.g., Prestwich, J Control Release. 2011 April 14, PMID 21513749; Perale, et al., Int J Artif Organs. (2011) 34(3):295-303; Suri, et al., Tissue Eng Part A. (2010) 16(5):1703-16; Khetan, et al., J Vis Exp. (2009) October 26; (32). pii: 1590; Salinas, et al., J Dent Res. (2009) 88(8):681-92; Schmidt, et al., J Biomed Mater Res A. (2008) 87(4):1113-22 and Xin, et al., Biomaterials (2007) 28:316-325.

As appropriate or desired, the engrafted or transplanted MSCs can be modified to facilitate retention of the MSCs at the region of interest or the region of delivery. In other embodiments, the region of interest for engraftment or transplantation of the cells is modified in order to facilitate retention of the MSCs at the region of interest or the region of delivery. In one embodiment, this can be accomplished by introducing stromal cell derived factor-1 (SDF-1) into the region of interest, e.g., using a linkage chemistry or integrated biodegradable matrix (e.g., Poly(D,L-lactide-co-glycolide (PLGA) beads) that would provide a tunable temporal presence of the desired ligand up to several weeks. MSCs bind to the immobilized SDF-1, thereby facilitating the retention of MSCs that are delivered to the region of interest for engraftment or transplantation. In other embodiments, integrating cyclic arginine-glycine-aspartic acid peptide into the region of interest can facilitate increased MSC binding and retention at the region of interest for engraftment or transplantation. See, e.g., Ratliff, et al., Am J Pathol. (2010) 177(2):873-83.

In some embodiments, at least about 0.25×10⁶ MSCs are injected into the subject. As appropriate, the number of MSCs injected into the subject may be at least about, e.g., 1×10⁴ cells, 2.5×10⁴ cells, 5×10⁴ cells, 7.5×10⁴ cells, 1×10⁵ cells, 2.5×10⁵ cells, 5×10⁵ cells, 7.5×10⁵ cells, 1×10⁶ cells, 2.5×10⁶ cells, 5×10⁶ cells, 7.5×10⁶ cells, 1×10⁷ cells, 2.5×10⁷ cells, 5×10⁷ cells, 7.5×10⁷ cells, or 1×10⁸ cells delivered in a single injection.

In various embodiments, the cells can be delivered at a concentration in the range of about 1×10⁶ cells/ml to about 1×10⁸ cells/ml, for example, in the range of about 5×10⁶ cells/ml to about 5×10⁷ cells/ml, for example about 1×10⁶ cells/ml, 5×10⁶ cells/ml, 1×10⁷ cells/ml, 5×10⁷ cells/ml or 1×10⁸ cells/ml.

A regime of treatment or prevention may involve one or multiple injections. For example, MSCs may be administered to the subject 1, 2, 3, 4, 5, 6, 7, 8, 9, 10, or more times, as appropriate. Multiple injections of MSCs may be administered to the same or different locations. For example, the MSCs may be administered in the peri-ocular space, e.g., in and/or around the lacrimal gland and/or in and/or around the meibomian glands of one or both eyes. Multiple injections of MSCs can be administered daily, weekly, bi-weekly, monthly, bi-monthly, every 3, 4, 5, or 6 months, or annually, or more or less often, as needed by the subject. The frequency of administration of the MSCs can change over a course of treatment, e.g., depending on how well the engrafted or transplanted MSCs establish themselves at the site of administration and the responsiveness of the subject. The MSCs may be administered multiple times over a regime course of several weeks, several months, several years, or for the remainder of the life of the subject, as needed or appropriate.

The total amount of cells that are envisioned for use depend upon the desired effect, patient state, and the like, and may be determined by one skilled within the art. Dosages for any one patient depends upon many factors, including the patient's species, size, body surface area, age, the particular MSCs to be administered, sex, scheduling and route of administration, general health, and other drugs being administered concurrently.

### 4. Monitoring Efficacy

Clinical efficacy can be monitored using any method known in the art. Measurable biomarkers to monitor efficacy include, but are not limited to, monitoring one or more of the physical symptoms of dry eye disease, for example, symptoms associated with keratoconjunctivitis sicca (KCS) and/or Meibomian Gland Dysfunction (MGD), including, e.g., loss, destruction and/or damage of the lacrimal gland tissue; loss, destruction and/or damage of the meibomian gland tissues; loss, destruction and/or damage of goblet cells; damage to the cornea (thickening of the corneal surface, corneal erosion, corneal ulceration, corneal neovascularization, corneal scarring, corneal thinning, and corneal perforation); insufficient, unproductive or non-lubricating tear production; eye irritation (e.g., dryness, burning, sandy-gritty sensations, itching, stinging, fatigue, pain, redness, pulling sensations); altered tear break up time (TBUT); alterations in aqueous tear production; and/or alterations in tear film osmolality. These parameters can be measured using any methods known in the art. For example, in various embodiments, tear production can be measured using one or more known techniques including without limitation Schirmer tear test (STT), phenol red thread testing and/or determining tear meniscus height.

Parameters for determining the extent, improvement and/or progression of MGD include without limitation tear interference image grades, tear evaporation rates, rose bengal staining scores, tear film breakup time (TBUT), meibomian gland and/or meibum expressibility, ocular dryness, lipid layer thickness. See, e.g., Goto, et al., Ophthalmology. (2002) 109:2030-2035; Goto, et al. Am J Ophthalmol. (2006) 142:264-270; and Ota, et al., Optom Vis Sci. (2008) 85:E795-E801.

Observation of the stabilization, improvement and/or reversal of one or more symptoms indicates that the treatment or prevention regime is efficacious. Observation of the progression, increase or exacerbation of one or more symptoms indicates that the treatment or prevention regime is not efficacious. For example, observation of the stabilization and/or increase of the mass, bulk, cell number and/or functionality of the lacrimal gland and/or meibomian glands after one or more administrations of MSCs indicates that the treatment or prevention regime is efficacious. Likewise, observation of an increase in lubricating tear production; tear production with normal concentrations and/or compositions of salts and/or lipids; a reduction in tear film osmolality; an increase in STT/phenol red thread testing; an increase in tear meniscus height; a reduction of eye irritation; a reduction in eye redness; and/or stabilization and/or reversal of corneal damage after one or more administrations of MSCs indicates that the treatment or prevention regime is efficacious.

In certain embodiments, the monitoring methods can entail determining a baseline value of a measurable biomarker or disease parameter in a subject before administering a dosage of the one or more active agents described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have a dry eye disease, nor are at risk of developing a dry eye disease. In such cases, if the value of the measurable biomarker or clinical parameter approaches the control value, then treatment is considered efficacious. In other embodiments, the individuals in the control population have not received prior treatment and have been diagnosed with dry eye disease. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

### 5. Cell Delivery Device

The invention further provides a cell delivery device useful to delivery of cells (e.g., MSCs) with improved accuracy and precision into and/or around a target tissue. The cell delivery device is comprised of a needle and a stop positioned along the length of the needle. When the needle is inserted into skin, the depth of delivery of the cells below the surface of the skin can be governed by the stop contacting the outer surface of the skin or by limiting passage of the needle through the conjunctiva if a transconjunctival delivery route is chosen as optimal for the subject, thereby facilitating the precise delivery of cells to a desired location into and/or around a target tissue.

Generally, the needle is a straight cylinder, but in certain instances can be curved if desired, so long as passage of the cells is not impeded. The diameter of the inner lumen or the gauge of the needle is sufficiently wide to allow ingress and egress of cells with minimal damage and/or shearing to the cells and sufficiently narrow to minimize or reduce trauma and/or damage to the skin and tissues pierced by the needle. In various embodiments, the needle has a gauge in the range of about 27 to 18 (based on the standard Stubs scale), for example, a gauge of 27, 26, 25, 24, 23, 22, 21, 20, 19 or 18. Accordingly, the diameter of the inner lumen can be in the range of about 0.19 mm to about 0.8 mm. The length of the needle will be sufficient to reach the target tissue from the outer surface of the skin or from the conjunctival surface (e.g., peri-lacrimal delivery can be accomplished by entering the superior conjunctival fornix). In various embodiments, needles for delivering cells into and/or around the peri-ocular space will have a length in the range of about 12 mm to about 65 mm, e.g., about 25 mm to about 50 mm, e.g., about 12, 15, 20, 25, 30, 40, 45, 50, 55, 60 or 65 mm. In various embodiments, needles for delivering cells into and/or around the lacrimal gland and/or into the stroma of the eyelid (e.g., particularly at the lid margin) will have a length in the range of about 12 mm to about 36 mm, e.g., about 25 mm. In embodiments using an adjustable stop, the stop can be slid along the barrel of the needle to determine the limit of passage and set the depth at which the point of the needle is able to penetrate. The bevel on the needle is of a shape and angle to cleanly puncture the skin or conjunctiva and allow delivery of the cells to the target tissue with minimal trauma to the tissue in the subject and minimal damage and/or shearing of the cells. In various embodiments, a needle having a standard bevel, a short bevel or a true short bevel can be used. The needles will generally be attached to a luer in order to conveniently connect with a syringe or catheter for delivery of the cells.

In various embodiments, the stop is adjustable, and the depth for needle insertion can be demarcated with one or more cross-wise (i.e., perpendicular to the length of the needle) score marks engraved into the needle. In other embodiments, the needle does not have score marks. In such cases, the stop can be positioned using an appropriate measuring device, e.g., a calipers a ruler. The engraved cross-wise scores can be along one side of the needle or rings banding the full circumference of the needle. The cross-wise scores can be engraved at uniform or non-uniform intervals along the length of the needle. In various embodiments, the needle has cross-wise score marks along the length of the needle uniformly spaced in the range of every 100 μm to 1500 μm, for example, every 100 μm, 200 μm, 300 μm, 400 μm, 500 μm, 600 μm, 700 μm, 800 μm, 900 μm, 1000 μm, 1100 μm, 1200 μm, 1300 μm, 1400 μm, 1500 μm. The stop is configured to slide along the length of the needle, and can be positioned at a score mark at a distance from the bevel that demarcates an appropriate depth for delivery of the cells into and/or around the target tissue, e.g., the lacrimal gland and/or the meibomian glands. In various embodiments, the internal lumen of the stop has a ridge to fit into the cross-wise score for stable positioning along the needle.

In various embodiments, the needle can have a permanently integrated (i.e., non-adjustable) stop that allows for delivery of cells at a pre-determined depth. The distance from the tip of the needle to the stop can vary according to the tissue intended to be targeted. For example, in some embodiments, a permanently integrated stop can be positioned at a distance from the bevel in the range of about 2 mm to 65 mm, for example, about 5 mm to 55 mm, for example, about 12 mm to about 36 mm, for example, about 25 mm to about 50 mm, for example, about 5, 12, 15, 20, 25, 30, 40, 45, 50, 55, 60 or 65 mm. It is noted that the design of the adjustable and non-adjustable stop on the cell delivery device can be adapted for delivery of cells to any target tissue of interest.

The needle can be composed of any appropriate material that is of sufficient strength and hardness to penetrate the skin and intervening tissues to deliver the cells to the target tissue and that is inert to the subject. Illustrative materials include without limitation metals and alloys (e.g., aluminum, silicon, titanium), plastics, and carbon fibers.

Off the shelf needles can be adapted to construct the cell delivery device by positioning a stop along the length of the needle. For example, the stop can be wrapped fully or partially around the needle, and scores or depth lines can be engraved cross-wise onto the outer surface of the needle.

The cell delivery device has a stop positioned along the length of the needle. The stop can be either fixed-in-place or adjustable, but its placement is sufficiently firm so as not to move upon encountering reasonable resistance upon contact with the outer surface of the skin. In some embodiments, the stop is constructed as a permanent fixture of the needle. In some embodiments, the stop encircles or snaps onto the needle, and can adjustably slide along the length of the needle, such that a clinician can place the stop to allow for insertion of the needle to an appropriate depth of tissue to deliver cells to the target tissue. Generally, the stop is positioned somewhere along the mid-length of the needle, without covering the bevel or egress lumen for the cells and without contacting the luer.

The stop can be any shape appropriate in order that the stop prevents or inhibits deeper insertion of the needle, once the stop contacts the outer surface of the skin. Illustrative shapes include a cylinder or disk. The stop can be round or elliptical, but may also be triangular, square or rectangular, depending on its use. The stop will generally have an inner lumen that is in contact with the needle, either partially or entirely hugging the circumference of the needle. The outer surface of the stop is of a width sufficiently wider than the needle such that upon contact with the outer layer of the skin, the stop prevents or inhibits deeper insertion of the needle. For example, the stop can be of a width that is at least about 1 mm to about 10 mm wider than the outer diameter of the needle, for example, at least about 1 mm, 2 mm, 3 mm, 4 mm, 5 mm, 6 mm, 7 mm, 8 mm, 9 mm, 10 mm, or wider, as appropriate. The end surface of the stop closest to the bevel of the needle, and which contacts the skin, can be either blunt or rounded. In certain instances, a rounded end surface is preferred in order to reduce or prevent trauma to the tissue contacted by the stop.

The stop can be composed of any material appropriate such that the stop functions for its intended purpose of governing the intended depth of insertion of the needle and that is inert to the subject. For example, in embodiments where the stop is permanent fixture of the needle, the stop can be of the same or different material as the needle. Illustrative materials appropriate for construction of the stop include without limitation metals and alloys (e.g., aluminum, silicon, titanium), plastics, rubber, and carbon fibers.

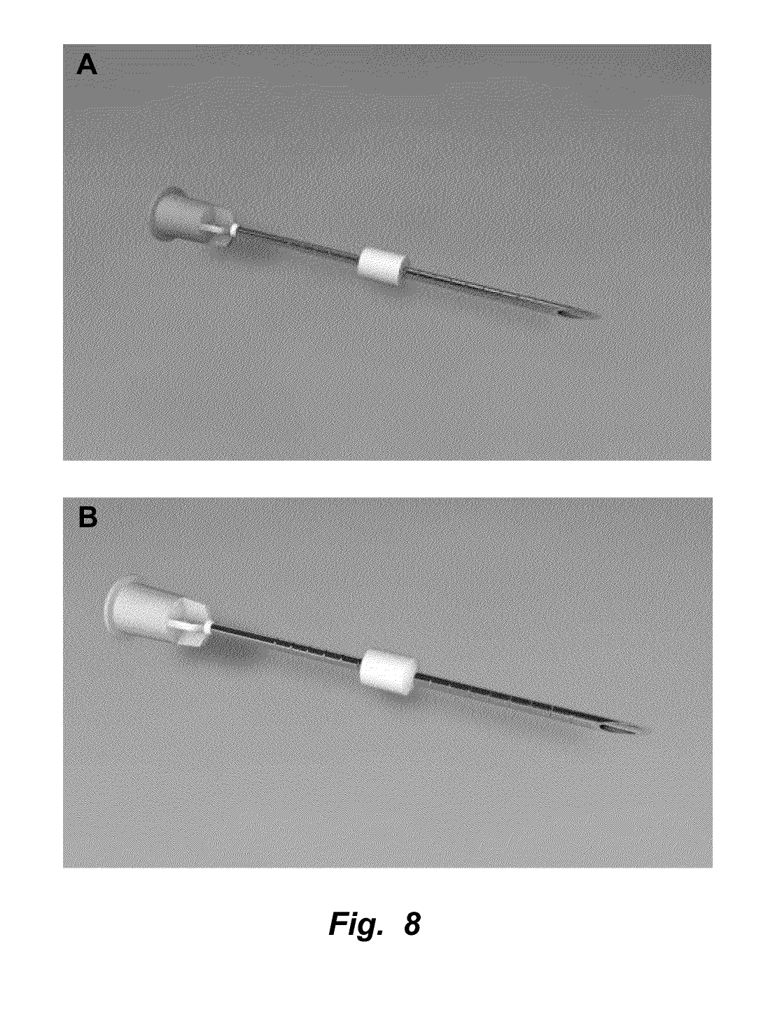

Illustrative cell delivery devices comprised of a needle and stop positioned along the need are shown in FIGS. 8A-B.

### 6. Kits

The invention further provides kits comprising a cell delivery device, as described herein, and MSCs. In various embodiments, the MSCs are derived from adipose or non-adipose tissue (e.g., derived from bone marrow, liver, lacrimal gland, and peri-ocular tissues). The MSCs may be contained in one or multiple vials. For example, in various embodiments, the kits comprise 2 or more vials. In some embodiments, the kits provide a minimum of 1×10⁴ cells, for example, at least about 2.5×10⁴ cells, 5×10⁴ cells, 7.5×10⁴ cells, 1×10⁵ cells, 2.5×10⁵ cells, 5×10⁵ cells, 7.5×10⁵ cells, 1×10⁶ cells, 2.5×10⁶ cells, 5×10⁶ cells, 7.5×10⁶ cells, 1×10⁷ cells, 2.5×10⁷ cells, 5×10⁷ cells, 7.5×10⁷ cells, or 1×10⁸ cells. The kits can further contain instructions for culturing and administering the cells, e.g., into and/or around the peri-ocular space, for example, into and/or around the peri-lacrimal space and/or into the stroma of the eyelid, particularly at the lid margin, into and/or around the meibomian glands. The embodiments of the cell delivery device are as described herein.

### EXAMPLES

The following examples are offered to illustrate, but not to limit the claimed invention.

### Example 1

### Peri-Ocular and Intra-Articular Injection of Canine Adipose Derived Mesenchymal Stem Cells: An In Vivo Safety, Imaging, and Migration Study

### Materials and Methods

Animals.

All animals in this study were used with approval by the Institutional laboratory Animal Care and Use Committee (IACUC) at the University of California, Davis. All protocols were also conducted with approval of the IACUC.

Ad-MSC Isolation and Culture.

MSCs were isolated from subcutaneous adipose tissue collected from the tail joint of seven clinically normal, 1 year old beagles as cultured as previously described. [Chung, et al., supra; Toupadakis, et al., American Journal of Veterinary Research (2010) 71(10):1237-1245] In brief, 10-13 g of adipose tissue was minced and rocked for at 37° C. for 2 hrs. in 50 mL of DPBS (Invitrogen, Carlsbad, Calif.) with 0.1% collagenase/1% bovine serum albumin (BSA) (Worthington, Lakewood, N.J.) followed by centrifugation to remove the lipid layer and repeated washes with DPBS. Cell pellets were resuspended with culture media [low-glucose DMEM (Mediatech, Manassas, Va.), 10% fetal bovine serum (HyClone Inc, Logan, Utah), and 1% Pen Strep (Invitrogen, Carlsbad, Calif.)], plated, and incubated at 37° C., 5% CO₂. Cells were passed at approximately 90% confluence.

Ad-MSC Phenotype.

Three of the ad-MSC lines were characterized using a panel of 12 monoclonal antibodies (including a negative control) using flow cytometry by combining 1×10⁶ Ad-MSCs with 25 μL of antibody for 30 min. at room temperature. (Table 1) The cells were then washed and pelleted twice followed by secondary labeling with 50 μL FITC conjugated horse anti-mouse IgG (Vector Laboratories, Carpenteria, Calif.) for 20 min. followed by washing. Flow cytometric measurement of 20,000 cells per antibody was performed using a FACScan flow cytometer (Becton Dickinson, San Jose, Calif.) with analysis using FlowJo software (Version 8.6.3, Tree Star Inc., Ashland, Oreg.).

TABLE 1
Monoclonal antibodies used to characterize Ad-MSCs

| Antigen | Clone | Source | Label | Expression |
|---|---|---|---|---|
| CD44 | S5 | B. Sandmater¹ | Adhesion | Positive |
| CD54 (ICAM-1) | CL18.1D8 | C. Smith² | Adhesion | Positive |
| CD90 (Thy-1) | CA1.4G8 | P. F. Moore³ | Stem cell marker | Positive |
| anti-feline CD1a | FE1.5F4 | P. F. Moore | Negative Control | Negative |
| CD3 | CA17.2A12 | P. F. Moore | T-cells | Negative |
| CD18 | CA1.4E9 | P. F. Moore | Leukocytes | Negative |
| CD34 | 1H6 | P. McSweeney¹ & R. Nash¹ | Hematopoietic lineags cells | Negative |
| CD45 | CA12.10C12 | P. F. Moore | Pan-leukocytes | Negative |
| CD49d (VLA-4) | CA4.5B3 | P. F. Moore | Adhesion | Negative |
| CD80 | CA24.5D4 | V. K. Affoiter³ | T-cell co-stimulator | Negative |
| CD86 | CA24.3E4 | V. K. Affoiter | T-cell co-stimulator | Negative |
| MHC class II | CA2.1C12 | P. F. Moors | MHC-II | Negative |

¹Fred Hutchinson Cancer Research Center, Seattle, WA.
²Houston, Tx.
³University of Callfomia, Davis.
Ad-MSCs were characterized by flow cytometry and positive for the adhesion markers CD 44 and CD 54 as well as the stem cell marker CD 90 (Thy-1). In conjunction, Ad-MSC population did not express CD 34 (hematopoietic lineage marker), CD 45 (pan-leukocytes), or MHC class II. Isolated Ad-MSC did not express T-cell markers: CD 3, CD 80, or CD 86 nor leukocyte marker CD 18 or adhesion marker CD49d.

DiD Labeling of Ad-MSC.

Ad-MSC were labeled according to the manufacturer's protocol with Vybrant DiD (Invitrogen, Carlsbad, Calif.). In brief, ad-MSC were washed twice with DPBS and removed from the flask with 5 mL of HyQtase (Hyclone, Logan, Utah) for 10 min. at 37° C., 5% CO₂ followed by pelleting and washing twice with DPBS and resuspension at 2×10⁶ cells/mL in warm, serum-free, DMEM (Invitrogen, Carlsbad, Calif.). The cells were then incubated for 15 min. at 37° C., 5% CO₂ with 5 μL/mL of DiD. Following labeling, the cells were washed and pelleted 3 times with DPBS and resuspended in DPBS in a sterile glass vial with protection from the light.

Engraftment or Transplantation.

Three canines received labeled cell injections of 2×10⁶ cells in 0.2 mL into the right peri-lacrimal and the region surrounding the 3rd eyelid (EL) gland on the 1st and 6th weeks of the study. Equivalent volume injections of DPBS were performed on the corresponding left regions of the canines An additional three canines received labeled cell injections of 66×10⁶ cells in 3 mL of DPBS into the right stifle joint on the 6th week of the study. All six canines received unlabeled injections of 2×10⁶ cells in 0.2 mL into the right peri-lacrimal and the region surrounding the 3rd EL gland. These injections were performed on a weekly basis throughout the 10 week study course except on the weeks that labeled cell injections were performed. Additionally, intra-articular injections of 5×10⁶ unlabeled cells in 0.5 mL were performed on all six canines on the 1st, 3rd, and 5th weeks of the study. Corresponding DBPS injections were performed on the left side at all injection time points.

In vivo imaging. All animals were initially anesthetized with 2.5-5 μg/kg of dexmedetomidine (WMI veterinary supply, Boise, Id.) and maintained with no more than 4 mg/kg of propofol (WMI veterinary supply, Boise, Id., 1-3 mL at 10 mg/mL) during imaging. A Maestro 2 imaging system (CRi, Woburn, Mass.) was modified to accommodate the dogs. In vivo imaging employed the “orange” filter set with excitation from 586-631 nm and 645 nm longpass emission filters. Emission images from 640-820 nm in 10 nm increments were taken at 1827 ms of exposure for lacrimal region images and 7500 ms for stifle region images with an F-stop setting of 8.4 in both cases and the focal plane set to zero. Image acquisition was set to 2×2 binning. The animals were held in the same positions for each image set throughout the course of the study. Maestro 2.10.0 software was used for all analysis. Identical background subtraction was performed on all lacrimal region images and all stifle region images and quantitative measurements of 10⁶ phot/cm²/s were used in all analysis to normalize for variations in animal size.

Necropsy.

Complete necropsies were performed by a board certified veterinary pathologist on all study animals within 15 min. after euthanasia to control for autofluoresence of tissues. Similarly sized samples of tissue were taken from over 65 locations including all major organs (e.g., kidney, liver, pancreas, spleen), lymph nodes, multiple locations along the GI tract, lung, heart, skeletal muscles, nervous tissues and bone. All tissues were thoroughly rinsed in 10% formalin to remove exogenous particulate matter such as blood or food prior to fixation and storage in 10% formalin in PBS.

Control Animals.

Tissue samples from healthy, non-species or age matched donors were collected within 2-4 hours after euthanasia. Tissue samples were rinsed thoroughly and fixed with 10% formalin in PBS prior to imaging.

Ex Vivo Imaging.

Each tissue source was imaged in a single scan from all nine animals (e.g. all six study dog colon samples and all three donor colon samples were imaged together) with 20,000 ms exposures per slice. Larger tissues such as orbits and stifle joints were imaged with appropriate vehicle injected controls and samples from each animal were imaged consecutively with identical image settings. Maestro 2.10.0 software was used for all analysis. Identical background subtraction was performed on all tissue images from study and control animals and quantitative measurements of 10⁶ phot/cm²/s were used in all analysis to normalize for variations sample size.

Statistical Analysis.

Data were analyzed using the Sigma Plot 11 software package (Systat Software, Chicago, Ill.). Analysis of variance (ANOVA) or Kruskal-Wallis ANOVA on ranks were used depending on the results of Chi-square normality tests to determine significance between multiple treatment groups. Paired and two-tailed Student's t-tests or Mann-Whitney rank sum tests were used to determine significance between two groups: */#=p<0.05, **/##=p<0.01, ***=p<0.001.

### Results:

In Vivo Fluorescence of Peri-Lacrimal Injected Ad-MSC.

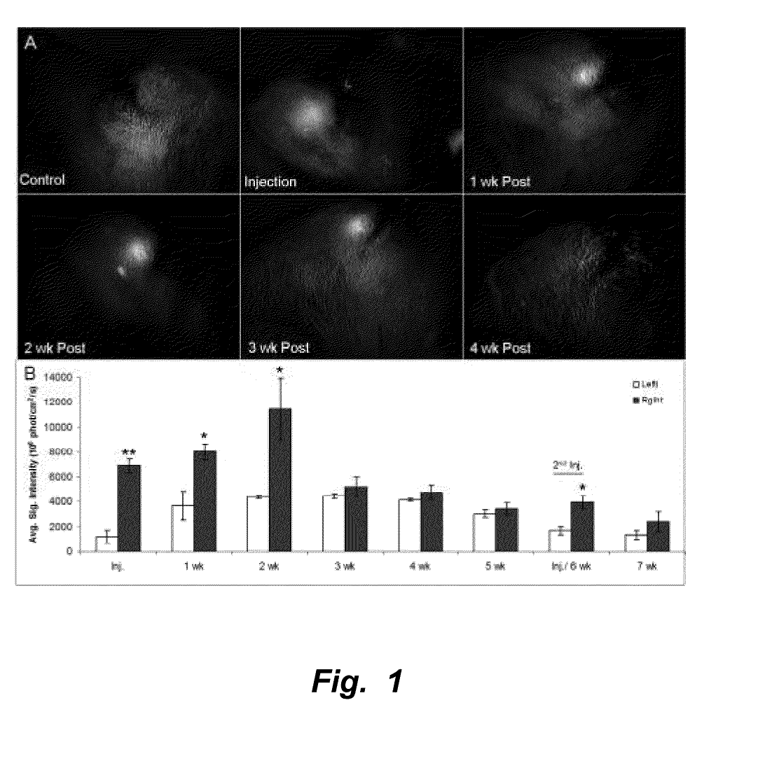

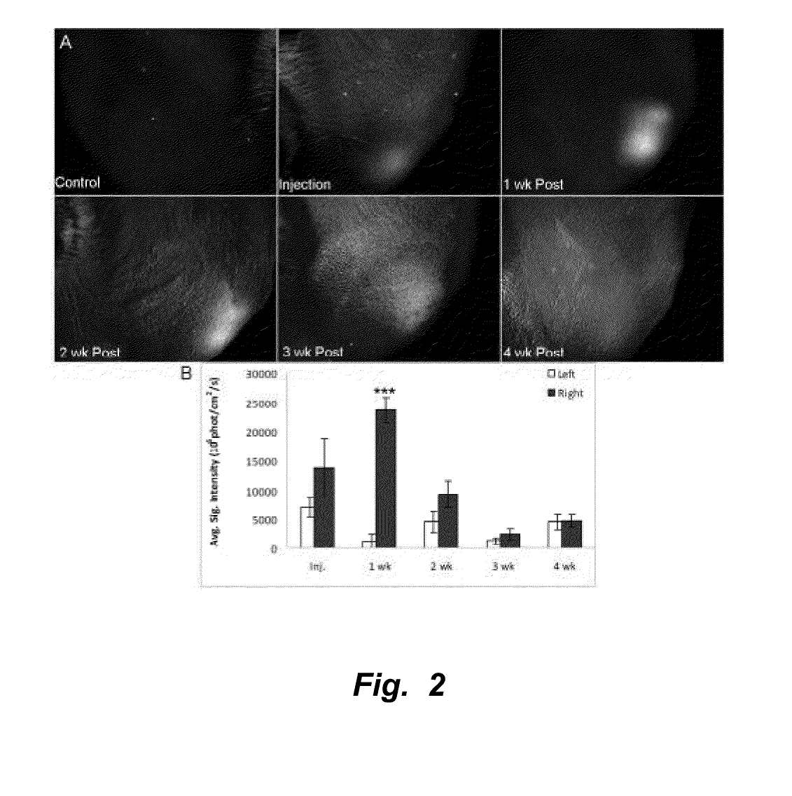

In vivo fluorescent detection of the DiD signal was used to determine the residence time of peri-lacrimal injected Ad-MSC. Fluorescent detection of the labeled Ad-MSCs can be seen in the peri-lacrimal region of the right side of the animals immediately following injection as compared to the vehicle injected control peri-lacrimal region on the left side of the animals (FIG. 1A) but was not observed in the region surrounding the 3rd eye lid (EL) gland. The peri-lacrimal signal persisted for up to 3 weeks in some animals with increasing intensity in the first 2 weeks followed by decreases in signal intensity in the 3rd week. The average signal intensity after injection was significantly brighter than the average signal intensity in the left peri-lacrimal region (FIG. 2B). The signal intensity remained significantly brighter than left side for 2 weeks before decreasing to an average level that was comparable to left side controls in the 3rd week. The right side peri-lacrimal signal levels were again significantly brighter than left side controls immediately following the second injection of DiD labeled cells in the 6th week. The signal was not significantly brighter one week later or throughout the remainder of the in vivo imaging time course.

In Vivo Fluorescence of Intra-Articular Injected Ad-MSC.

The fluorescent signal from DiD labeled Ad-MSCs was detectable in both the lateral and medial (FIG. 2A) sides of the right hind-limb following injection. In vivo detection of the fluorescent signal was discernable from background for up to 2 weeks after injection in some animals. Peak fluorescent intensity was observed one week after injection with an average signal intensity of 23,650×10⁶ (phot/cm²/s) which was significantly brighter than left side controls.

Ex Vivo Fluorescence of Peri-Ocular Injected Ad-MSC.

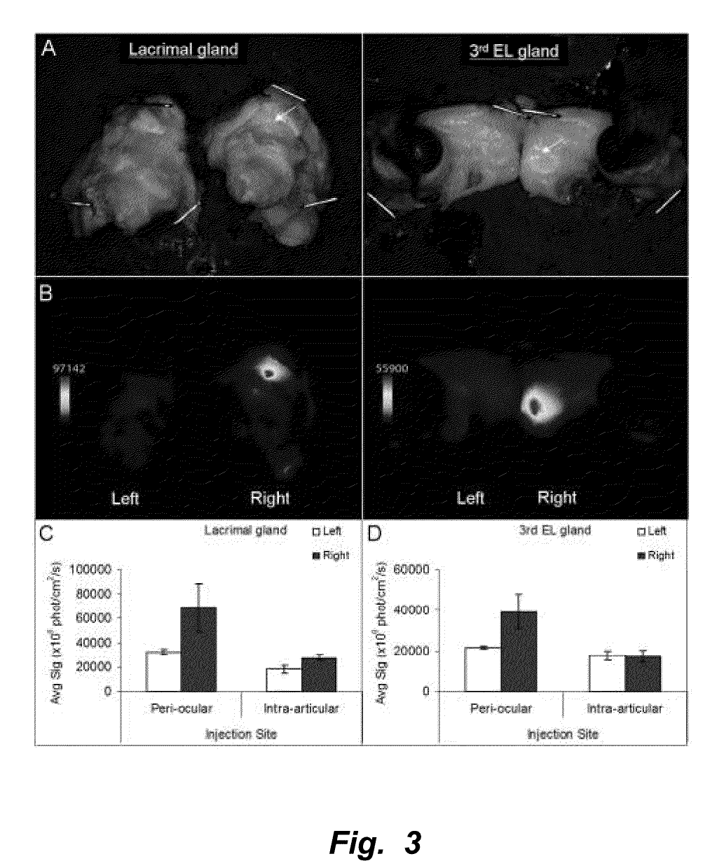

Images of the surrounding orbital tissue ex vivo demonstrate the persistence of fluorescence from DiD labeled Ad-MSCs more than 4 weeks after injection in both the peri-lacrimal region and around the 3rd EL gland (FIG. 3A). Heat map images demonstrate the concentration of the Ad-MSCs in the injection sites. The average signal intensity in the peri-lacrimal region of the right eye was more than twice the average signal intensity of the left eye as well as the right and left eyes of intra-articular injected animals (FIG. 3C). Similarly, the average signal intensity in the region around the 3rd EL gland of the right eye was nearly twice that of the left eye and more than twice the signal in the right and left eyes of intra-articular injected animals (FIG. 3D).

Ex Vivo Fluorescence of Intra-Articular Injected Ad-MSC.

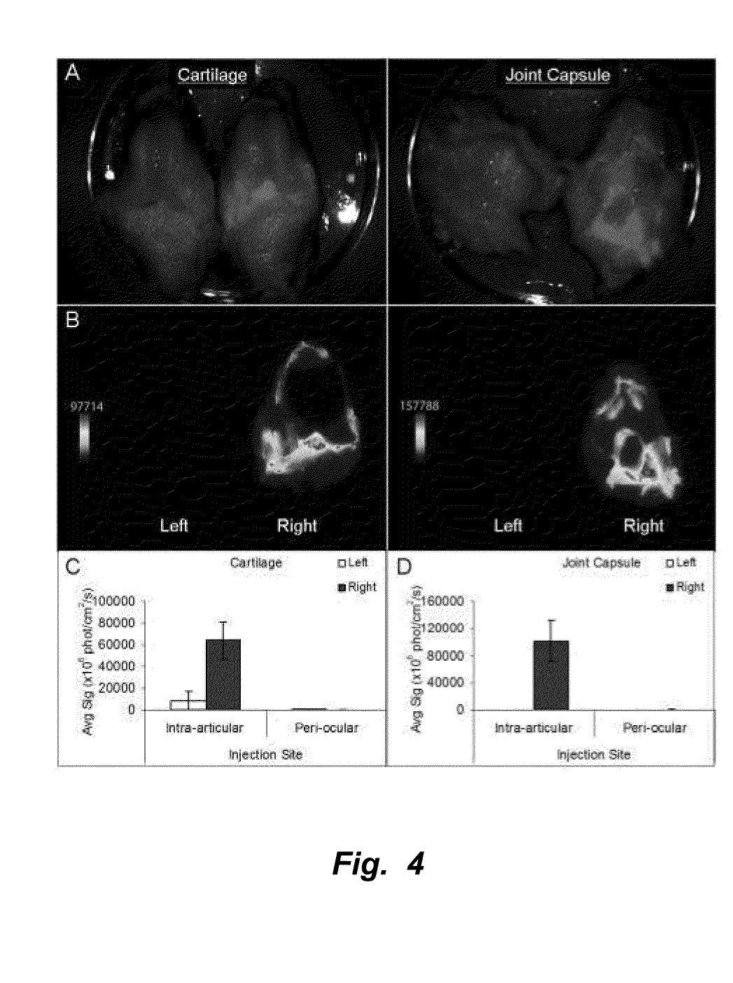

Ex vivo images of the left and right stifle cartilage and joint capsule from the hind-limbs of intra-articular injected animals demonstrate the persistence of fluorescence from labeled Ad-MSCs over 4 weeks after injection (FIG. 4A). Heat map images demonstrate the localization of the cells around the synovium of the joint (FIG. 4B). The average signal intensity in the cartilage surrounding the stifle joint was more than 7 times greater than the average signal intensity from the left stifle joint cartilage and nearly 70 times greater than the average signal intensities from the left and right stifle joints of peri-ocular injected animals (FIG. 4C). Even brighter, the average signal intensity in the joint capsule of the right stifle joint of intra-articular injected was more than 150 times greater than the joint capsule of either the left stifle joint of intra-articular injected or either stifle joint of peri-ocular injected animals (FIG. 4D).

Migration of Ad-MSC.

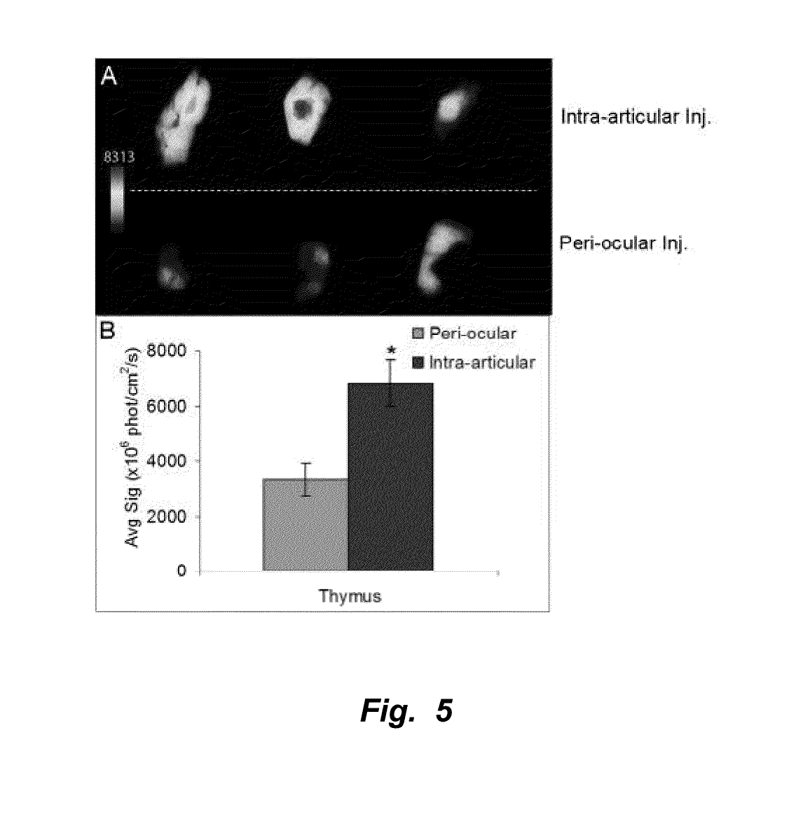

To investigate migration of the Ad-MSCs following engraftment or transplantation, ex vivo imaging over 65 tissues was performed. Significant results were found in the thymus, and the tissues of the gastro-intestinal (GI) tract including: stomach, duodenum, jejunum, and colon. The majority of the tissues imaged including lung, did not contain a fluorescent Ad-MSC population. The thymus of intra-articular injected animals contained a significant population fluorescent Ad-MSCs compared to peri-ocular injected animals (FIG. 5A). The average signal intensity in the thymus of intra-articular injected animals was more than twice the average signal intensity from the peri-ocular injected animals.

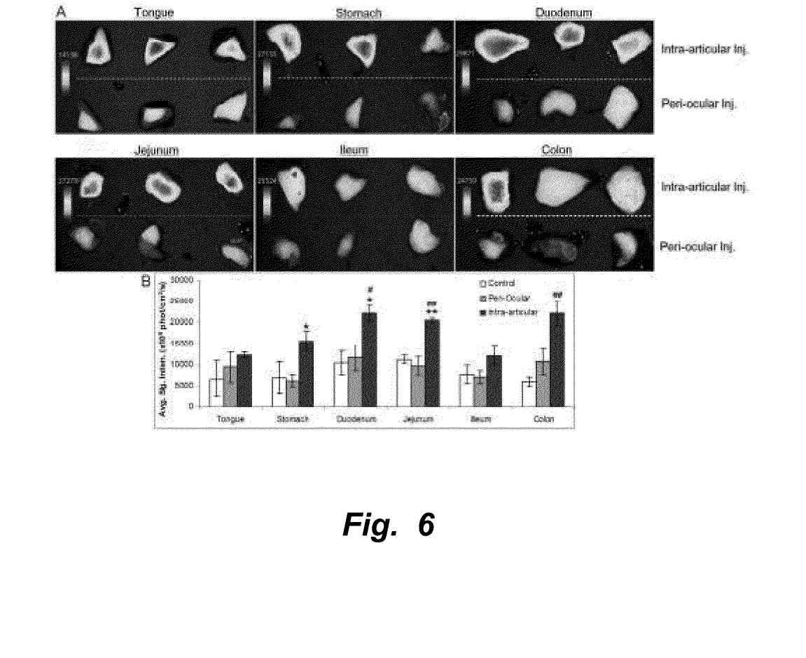

Ex vivo heat map images of the GI tract demonstrated the engraftment of the Ad-MSCs (FIG. 6A). Overall, engraftment was highest in the intra-articular injected animals. Significant levels of fluorescence compared to peri-orbital injected animals were found in the stomach, duodenum, and jejunum. Similarly, significant levels of fluorescence compared to non-injected donor controls were found in the duodenum, jejunum, and colon (FIG. 6B).

### Discussion:

Signal intensity is strongly affected by the ability of the fluorescence to penetrate the layers of tissue between the light source and cell source during excitation and the cell pool and the detector during emission. This is clearly demonstrated by the diminished signal intensity observed in the in vivo images (FIG. 1B) compared to the brighter signals measured from ex vivo images more than two weeks later (FIG. 3B). Additionally, hyperpigmentation was noted during the later time points of our study that reduced the ability to detect fluorescence. Changes in environmental exposure and chronic contact dermatitis caused by repeated shaving are well documented to induce this type of hyperpigmentation in canines [Melman, Skin Diseases of Dogs and Cats. 1994, Potomac, Md.: DermaPet Inc.; McKeever, et al., A Color Handbook of Skin Diseases of the Dog and Cat. 2nd ed. 2009, London: Manson Publishing; Patterson, Skin Diseases of the Dog. 1998, Oxford: Blackwell Science Ltd, Campbell, The Pet Lover's Guide to Cat & Dog Skin Diseases. 2006, St. Louis, Mo.: Elsevier Saunders; and Gross, et al., Skin Diseases of the Dog and Cat: Clinical and Histopathological Diagnosis. 2nd ed. 2005, Oxford: Blackwell Sciences]. Data trends in the left, vehicle injected control side of the animals demonstrate an initial increase in signal intensity that may be the product of increased removal of fur following the initial shaving prior to the 1st injection. The average signal intensity begins to decline in the 4th week and steadily declines throughout the remainder of the study (FIG. 1B). The steady decline in average signal intensity correlates with the increased skin pigmentation. This also accounts for the relatively low signal intensity following the second injection and the inability to detect the signal thereafter.

While successful survival of Ad-MSC following engraftment or transplantation with similar protocols is well documented [Hong, et al., Current Opinion in Organ Transplantation, (2010) 15(1):86-91; Mizuno, Journal of Nippon Medical School (2009) 76(2):56-66; Nakao, et al., The American Journal of Pathology, (2010) 177(2):547-554], it is also possible that diminution of the signal was due to in part to Ad-MSC lysis or phagocytosis following engraftment or transplantation. In the event of cell lysis, lipophilic dyes, such as DiD, are released and may bind surrounding cells. The migration of the cells and localized fluorescence demonstrate that this is unlikely in our study. In the event that the dye migrated systemically the fluorescence would be diffuse and no longer localized. In addition, the fluorescent dye would be present in either the lymphatic vessels or blood vasculature. Labeling of specific organs was found in the GI tract and the thymus but no labeling of lymphatic vessels, local lymph nodes or distal lymph nodes. No labeling of the blood vasculature was observed which demonstrates that cell lysis on a large scale is unlikely. Absence of labeling in the lymph nodes also demonstrates that phagocytosis by macrophages and innate or adaptive immune responses to the cells are unlikely. Additionally, MSCs have been shown to evade immune detection and are even immunomodulatory in nature [Liechty, et al., Nat Med, (2000) 6(11):1282-1286, Ramasamy, et al., Cellular Immunology (2008) 251(2):131-136; Riordan, et al., J Transl Med (2009) 7(29); and Kang, et al., Stem Cells and Development, (2008) 17(4):681-694]. The successful migration of the Ad-MSCs (FIGS. 5 & 6) following engraftment or transplantation demonstrates that at least a portion of the injected MSCs survived extravasation and engraftment into alternative tissue sites.

Another possible explanation for the diminution of the signal is cell migration. MSCs from multiple sources have been shown to migrate following engraftment or transplantation in several animal models, including in canine and human patients [Schäffler, et al., Stem Cells (2007) 25(4):818-827; Liechty, et al., Nat Med (2000) 6(11):1282-1286; and Barbash, et al., Circulation (2003) 108(7):863-868]. Use of iron-oxide (FeO) labeled Ad-MSCs demonstrated migration of some of the cells from the initial, peri-lacrimal injection site. The present data shows the contribution of the MSCs to the thymus. The contribution of autologous, labeled MSC to the thymic epithelium to support lymphopoiesis has been previously demonstrated in mice [Suniara, et al., The Journal of Experimental Medicine (2000) 191(6):1051-1056] as has the contribution of human MSCs to the thymus of sheep following engraftment or transplantation [Liechty, et al., Nat Med (2000) 6(11):1282-1286]. The primary mechanism for MSC inhibition of T cell response is thought to be through down regulation of activated T cell proliferation [Ramasamy, et al., Cellular Immunology (2008) 251(2):131-136]. Combined with previous findings, the contribution of the MSCs to the thymus of intra-articular injected dogs (FIG. 5) suggests that the engrafted or transplanted MSCs are functioning in an immunomodulatory fashion.

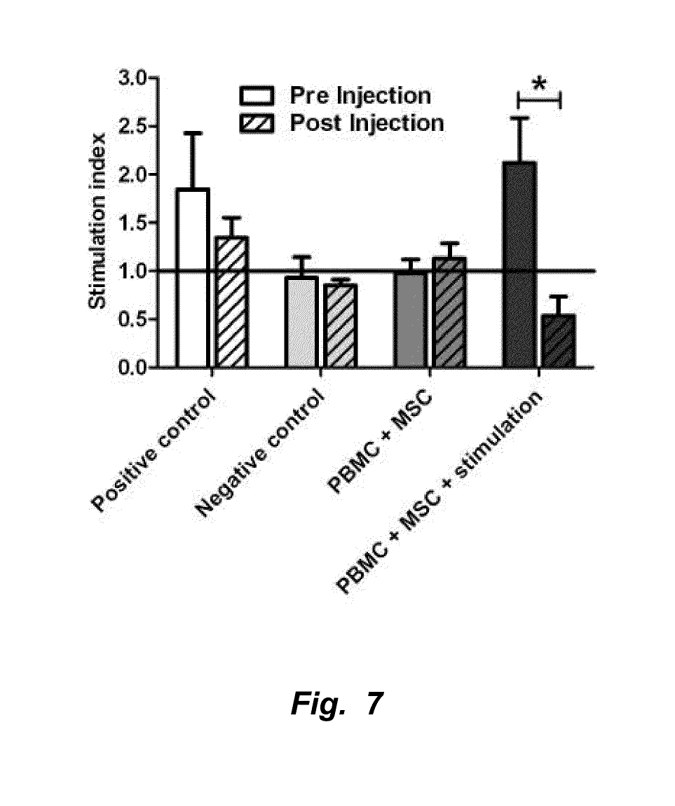

Migration of the Ad-MSC throughout the GI tract was also found (FIG. 7). Previous investigations with Bone marrow derived MSCs demonstrate their migration to the intestine [Jiang, et al., Nature (2002) 418(6893): p. 41-49; Colletti, et al., Stem Cell Research (2009) 2(2):125-138]. Furthermore, endothelial progenitor cells (EPCs), which have been shown to originate from a common progenitor source with MSCs [Vodyanik, et al., Cell Stem Cell, (2010) 7(6):718-729], contribute to the stem cell pool and supporting myofibroblast niche in the small intestine following engraftment or transplantation into fetal sheep [Wood, J. A., Human endothelial progenitor cells: A novel and promising cellular therapy for regenerative medicine, in School of Medicine. 2009, University of Nevada, Reno. p. 143.]. Furthermore, MSCs have been used to regenerate small intestine tissue following engraftment or transplantation on scaffolds [Hori, et al., Journal of Surgical Research (2002) 102(2):156-160; Nakase, et al., Tissue Engineering (2006) 12(2):403-412]. However, the relative level of engraftment throughout the entire GI tract by Ad-MSC in a large animal model has been quantified herein for the first time.

Significant results were more apparent in the intra-articular injected animals because they received 66 million labeled Ad-MSC in one bolus dose as compared to the 8 million Ad-MSC injected into the peri-orbital region. Engraftment of Ad-MSC from intra-articular injected animals was statistically higher in the colon compared to non-injected control animals but not peri-ocular injected animals. Interestingly, engraftment in peri-ocular injected animals was over 40% higher as compared to control animals (FIG. 7B). The data trend suggests that peri-ocular injection of Ad-MSCs may result in engraftment in the colon as well but larger cell doses would be needed to confirm these results. The reason for migration to these sites remains unknown but is possibly due to the cytokine profile in the healthy peri-lacrimal and intra-articular regions which may motivate migration of the cells away from these sites. The native cytokine profile of the highly proliferative GI tract may motivate engraftment.

In contrast to previous studies where migration to the lung was observed following intravenous injection of MSCs [Liechty, et al., Nat Med (2000) 6(11):1282-1286; Barbash, et al., Circulation (2003) 108(7):863-868., Chin, et al., Nuclear Medicine Communications (2003) 24(11):1149-1154], peri-orbital and intra-articular injection of MSCs did not lead to engraftment in the lungs. The stifle skin immediately superior to the right stifle injection site was found to be fluorescent during ex vivo examination most likely due to spreading of cells along the injection path. Overall, significant MSC engraftment was not found in any of the other tissue locations from over 65 tissues sampled from each animal.

In conclusion, the persistence of Ad-MSC in the healthy peri-orbital and intra-articular injection sites in combination with the immunomodulatory nature of Ad-MSCs demonstrate their potential for the treatment of both DED and RA diseases. Increases in inflammatory cytokines associated with DED [Solomon, et al., Investigative Ophthalmology & Visual Science, (2001) 42(10):2283-2292] and RA [Feldmann, et al., Annual Review of Immunology (1996) 14(1):397-440] and various chemokines may improve MSC function and engraftment [Chamberlain, G., et al., Stem Cells (2007) 25(11):2739-2749]. While studies are ongoing on the application of Ad-MSC to RA diseases [Gonzalez-Rey, et al., Annals of the Rheumatic Diseases (2010) 69(00:241-248], further studies are needed to fully elucidate the impact of MSC therapy in an immunomodulatory capacity on autoimmune disease such as DED and RA. In conjunction with DED and RA related autoimmune diseases, the preferential engraftment of Ad-MSCs to the GI tract demonstrates the potential for the treatment and repair of a host of autoimmune as well as degenerative diseases and disorders of the gut.

### Example 2

### Peri-Ocular and Intra-Articular Injection of Canine Adipose Derived Mesenchymal Stem Cells: An In Vivo Safety, Imaging, and Migration Study

This example provides patient follow-up data showing that administration of MSC in a canine subject produced no adverse events. Moreover, the tear values have stayed elevated in the treated eye.

### Technical Procedural Outline:

Initial Ophthalmic examination and diagnosis of primary bilateral KCS with a Schirmer Tear Test (STT) value greater than 2 and less than 10 (i.e., >2 & <10).

First MSC injection. Blood sample for mixed lymphocyte or leukocyte reaction (MLR) (immune status test). Patient was sedated and the MSC's injected into the determined locations (lacrimal gland and gland of the 3rd eyelid). This was done at week 0, week 3 and week 8.

Before initiating injections of MSC, administration of Cyclosporine (or other immunosuppressant) was stopped in one eye for 2 weeks and supplemented with artificial tears three times daily. The prescription medication was re-initiated one week prior to the first MSC injection.

Ophthalmic examinations are re-checked and Schirmer tear tests (STT) were performed at the following time points.
Week 0 (First MSC injection)
Week 3 (Second MSC injection)
Week 4 (7 days post-injection). The prescription immunosuppressant was discontinued in one eye. The patient was rechecked on Day 2 and Day 5 of Week 4 and again on Day 3 of Week 5 (i.e., 10 days after discontinuing the prescription).
Week 8, (3rd MSC injection) the eye was evaluated and it was determined if the prescription immunosuppressants needed to be re-initiated.
Week 10; prescription immunosuppressant discontinued if it was restarted at Week 8 and reassessed on Day 2 and Day 5 of Week 10, and again on Day 3 of Week 11 (i.e., 10 days after discontinuing the prescription).
Week 16. A second blood sample was taken for mixed lymphocyte or leukocyte reaction (MLR).

The results are presented in Table 2:

TABLE 2

| | STT | STT | |
|---|---|---|---|
| DAY | OD | OS | COMMENTS |
| −188 | 4 | 2 | RDVM visit: pre-CsA: started CsA 2% OU BID |
| −184 | 12 | 13 | RDVM visit |
| −49 | 23 | 21 | first VMTH visit: discontinued CsA OD at this visit |
| −42 | 8 | 18 | off CsA OD for 1 week; started back on CsA OD |
| −30 | 17 | 16 | after visit surgery day for adipose harvest |
| 0 | 16 | 17 | injection day 0 |
| 7 | 19 | 17 | 1 week |
| 19 | 17 | 23 | 3 weeks |
| 28 | 19 | 20 | 4 weeks |
| 42 | 19 | 20 | 6 weeks |

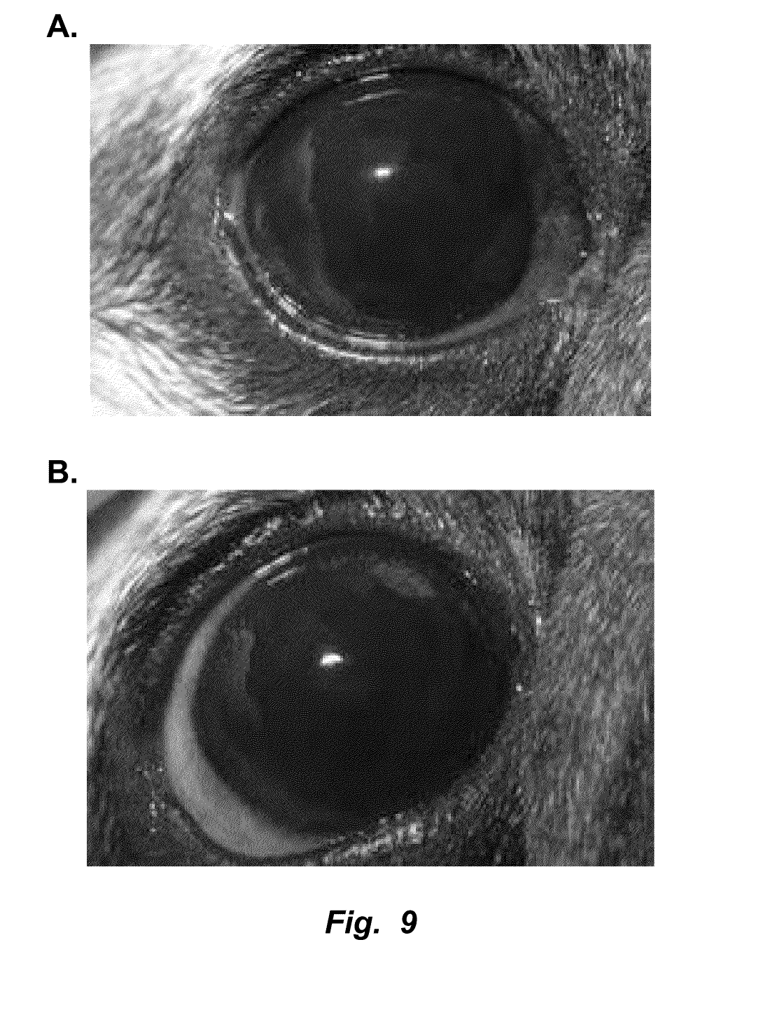

The treated canine subject had no adverse events and tear values stayed elevated in the treated eye. See, FIGS. 9A-B.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preventing, delaying, reducing or inhibiting a tear film disorder in a subject in need thereof comprising engrafting or transplanting in the subject mesenchymal stem cells (MSC).

2. The method of claim 1, wherein the MSCs are adipose-derived mesenchymal stem cells (Ad-MSC).

3. The method of claim 1, wherein the MSCs are derived from a non-adipose tissue.

4. The method of claim 3, wherein the non-adipose tissue is selected from the group consisting of bone marrow, liver, lacrimal gland, and peri-ocular tissues.

5. The method of claim 1, wherein the MSCs are selected from autologous to the subject, syngeneic to the subject, allogeneic to the subject and xenogeneic to the subject.

6-8. (canceled)

9. The method of claim 1, wherein the MSCs are administered systemically or peri-ocularly.

10. (canceled)

11. The method of claim 1, wherein the tear film disorder is keratoconjunctivitis sicca (KCS).

12-14. (canceled)

15. The method of claim 1, wherein the MSCs are engrafted or transplanted in and/or around the lacrimal gland.

16. The method of claim 1, wherein the tear film disorder is Meibomian Gland Dysfunction (MGD).

17. The method of claim 16, wherein the MGD is selected from the group consisting of posterior blepharitis, meibomian gland disease, meibomitis, meibomianitis, and meibomian keratoconjunctivitis.

18-19. (canceled)

20. The method of claim 16, wherein the MSCs are engrafted or transplanted into the stroma of the eyelid.

21-22. (canceled)

23. The method of claim 21, wherein the MSCs are engrafted or transplanted in and/or around the conjunctiva.

24. The method of claim 1, wherein at least 0.25×10⁶ MSCs are administered to the subject.

25. The method of claim 1, wherein the MSCs are administered to the subject multiple times.

26. The method of claim 25, wherein the MSCs are administered to the subject monthly.

27. The method of claim 1, wherein the subject is a mammal selected from a human and a canine.

28. (canceled)

29. The method of claim 1, wherein the subject has an autoimmune disease or has a predisposition to developing an autoimmune disease.

30. The method of claim 29, wherein the autoimmune disease is selected from the group consisting of rheumatoid arthritis, Wegener's granulomatosis, systemic lupus erythematosus (SLE), Sjögren's syndrome, scleroderma, primary biliary cirrhosis, diabetes, Vogt-Koyanagi-Harada Syndrome (VKH Syndrome) and Stevens-Johnson syndrome.

31. The method claim 1, wherein the MSCs express CD44 and CD90 and do not express CD34, CD45, CD80, CD86 or MHC-II.

32. (canceled)

33. The method of claim 1, wherein the engrafted or transplanted MSCs are not derived from and do not comprise cells from the lacrimal gland.

34. A cell delivery device, comprising a needle and a stop positioned along the length of the needle.

35-36. (canceled)

37. A kit comprising mesenchymal stem cells (MSCs) and the cell delivery device of claim 1.

38. (canceled)
